(19) United States
(12) Patent Application Publication
(10) Pub. No.: US 2006/0063971 A1
(43) Pub. Date: Mar. 23, 2006

(54) Method for attaching prosthetic penile attachments/extensions

(76) Inventor: Donald Hill, Mount Pleasant, SC (US)

Correspondence Address: EDWIN TARVER, 16830 Ventura Blvd., SUITE 360, Encino, CA, 91436, US

(21) Appl. No.: 11/204,839

(22) Filed: Aug. 15, 2005

Related U.S. Application Data
(60) Provisional application No. 60/601,479, filed on Aug. 14, 2004.

(51) Int. Cl. A61F 5/00

(52) U.S. Cl. 600/38

(57) ABSTRACT

The present invention is a prosthetic penile attachment/extension device that provides a more comfortable and faster method of pulling it over an erect or semi-erect male phallus to secure it in place. The device comprises a generally tubular shaped body having an exterior end and an interior end; an air passage fitted in between the exterior and interior ends; a head portion on the exterior end; and a sidewall of uniform or varying thickness that disposes the interior end spatially and coaxially inside the exterior end. A male user with an erection inserts his penis into the open end of the device all the way to the head portion. The user then seals the air passage with either a fitted, condom-like cap, common condom, or stopper-like cap. The seal will create an ambient air pressure vacuum that is sufficient to prevent the device from slipping or falling off during use.

### CROSS-REFERENCE TO RELATED APPPLICATION

This is a utility patent application which claims benefit of U.S. Provisional Application No. 60/601,479 filed on Aug. 14, 2004.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates to an improved prosthetic device for attachment and use with a male phallus that provides aid during sexual intercourse, and more particularly, to a method of applying and securing a prosthetic device over a male phallus.

Sexual fulfillment can play an important role in the relationship between a man and a woman. Experts generally agree that it is important for both parties in a relationship to attain fulfillment while engaging in sexual acts with their partner. Physical and psychological factors are involved, one of which can be the relative size of the male phallus. Many men seek to increase the size of their penis to feel better about their sexual potency and/or to provide more sexual stimulation for their partners during intercourse. Men have even resorted to various surgical procedures which are expensive and can result in serious medical complications and deformity.

Devices such as extenders have been made available in the market that are worn over an erect male penis to artificially and temporarily increase exterior length and circumference. There are however many problems with the design and function of these various devices. Many are not capable of adequately securing the device in place. Others are difficult or even impossible to put on. Some devices rely on external straps or other devices that can interfere with sexual intercourse and detract from the natural and aesthetic appearance of the user. Many extenders currently on the market rely solely on friction and are either too small in interior diameter making them impossible or very difficult to get on, while others are too large in interior diameter and do not provide a snug fit so that they are prone to slipping or falling off during use. Even when properly sized to the size of the wearer, these extenders are also very difficult to pull over the male phallus because they do not have a mechanism that allows the air in the sleeve to escape. The air must work its way around the base of the shaft, which is difficult if the sleeve fits snugly as it should. Some extenders currently available in the market rely on external straps secured either around the wearer's waist or testicles to hold the extender in place. These extenders can be difficult to put on, the various straps and retaining devices can become a hindrance during use, and they are not aesthetically appealing.

Moreover, in such extenders, it is difficult to get all of the air out of the sleeve, and a pocket of air will most likely remain within the device. The existence of this trapped air allows the extender to slip during use as the air is alternatively compressed within the extender during inward thrusting, then expanded during withdrawal. This trapped air can also create a soft spot in the interior of the device that will allow it to kink or bend in an unnatural and displeasing manner during use.

A number of devices have been developed in the art to temporarily and artificially increase the length and/or width of the male phallus in order to enhance sexual intercourse.

U.S. Pub. No. 20040171911 to Zurita describes a pneumatic penile prosthesis device adapted to be worn over the penis with the visual appearance of an erect penis. The device is intended to allow males with erectile dysfunction to engage in sexual intercourse. The device comprises two parts that are generally of the same length, tubular shaped, and preferably made of a semi-rigid plastic material. A check-valve permits selective de-pressurization of an annular space formed around the penis inside the prosthesis, thereby applying vacuum pressure to maintain the prosthesis in position. Although similar in that both rely on vacuum pressure to retain the devices in place, there are several significant distinctions between this device and the present invention. US Patent No. 2004/0171911 (Zurita) is intended to be used by males suffering from erectile dysfunction. The present invention is intended to be used by males who can obtain and maintain an erection, and is intended to enhance sexual intercourse by adding girth and length to the wearer's erection. The Zurita device must be made of a rigid or semi-rigid material to compensate for the lack of rigidity in the wearer's penis. The present invention is made of a softer and more pliable material such as soft PVC plastic or silicone since it not intended to compensate for a lack of rigidity in the wearer's natural penis. The Zurita patent also relies on a two-piece phallus. This is required so the wearer can manually pull the flaccid penis through a constriction ring at the base of the device, and then enclose it within the device when the top half of the prosthesis is screwed into place. The present invention requires no constriction ring or two-piece construction since it relies on the rigidity inherent within an erect male penis to enable the wearer to pull the extension/prosthesis over the phallus. The Zurita device also requires the use of an external vacuum pump to evacuate air out of the device. A one-way check valve maintains the air vacuum within the device. The current invention does not require an external pump to create a vacuum since the air within the device is forced out as the wearer pulls the device over his erect penis.

In U.S. Pat. No. 6,749,558 to Brintle, a marital aid kit is disclosed. It comprises a plurality of semi-rigid penile extensions of varying sizes, a retainer harness attached to the penile extension to keep said penile extension in place on the male body and a sizing chart to assist the user in selecting the appropriate size penile extension for proper fit.

U.S. Pat. No. 5,868,137 to Brown discloses a penis support device to help provide stiffness to the penis for those men who have trouble getting or maintaining an erection for the purpose of having satisfactory sexual intercourse. The device is generally tubular in shape, having a slit running the length of the tube.

Although the above devices and similar ones in the art disclose inventions designed for similar purpose as that of the present invention, there are structural distinctions, and they don't provide an easily applied and effective method of securing the prosthetic device to keep it from slipping or falling off during use.

Therefore, there is a need for a device that provides a more comfortable and faster method of pulling a prosthetic penis attachment/extension over an erect male phallus to secure it in place.

It is an object of the present invention to overcome the shortcomings in the current technology and to provide a method of applying and securing a prosthetic penile attachment/extension over an erect male phallus that creates an ambient air vacuum to secure it in place and keep it from slipping or falling off during use.

A further object is to provide a method of applying and securing a prosthetic penile attachment/extension over an erect male phallus that eliminates soft spots caused by trapped air pockets.

A further object is to provide a method of applying and securing a prosthetic penile attachment/extension over an erect male phallus that enables it to remain in place even if the wearer loses his erection.

Finally, it is an object of the present invention to provide a method of applying and securing a prosthetic penile attachment/extension over an erect male phallus that securely holds in place without the use of any retaining strap or other external devices, which could cause discomfort to the wearer and/or his partner, while it presents a more natural look and feel. These and other objects of the present invention will become better understood with reference to the appended Summary, Description, and claims.

### SUMMARY

The present invention provides a new and more effective way to attach a prosthetic penile attachment/extension device over an erect or semi-erect male phallus to enhance sexual intercourse. The invention provides a design that incorporates an air passage at the end of the attachment, which allows air to escape the interior of the attachment as the wearer pulls it over his phallus.

The wearer then closes the air passage by one of various methods. The condom-like cap feature of this invention is one of the methods that can be used to seal the air passage. A common condom, plug, or check valve are also some of the possible, alternate embodiments that can be used. Thus, by using these embodiments for sealing the air passage, an ambient air pressure vacuum is formed within the attachment that keeps the prosthetic penile attachment from falling off or slipping during use.

The usage of an air passage and means for sealing the air passage make this penile attachment device easier to put on, keeps it on during use, and makes it more comfortable and aesthetically pleasing for the user. Different materials, shapes, and sizes, including vibrating motors to provide additional stimulation, can also be adopted for the present invention depending on the current demands of the market.

### BRIEF DESCRIPTION OF THE FIGURES

FIG. 1 is a lateral cross sectional view of a prosthetic penile attachment/extension with an air passage at the end of the device in accordance with the present invention.

FIG. 2 is a front view of the prosthetic device as shown in FIG. 1.

FIG. 3 is a back view of the prosthetic device as shown in FIG. 1.

FIG. 4 is an illustration showing three different methods of sealing the air passage in accordance with the present invention.

FIG. 5 is a perspective view of the prosthetic penile attachment/extension in accordance with the present invention.

### FIGURES—REFERENCE NUMERALS

10 . . . Prosthetic Penile Attachment/Extension Device
11 . . . Air Passage
12 . . . Head
13 . . . Interior End
14 . . . Exterior End
15 . . . Sidewall
16 . . . Open End
17 . . . Condom-like Cap
18 . . . Condom
19 . . . Stopper-like Cap

### DETAILED DESCRIPTION

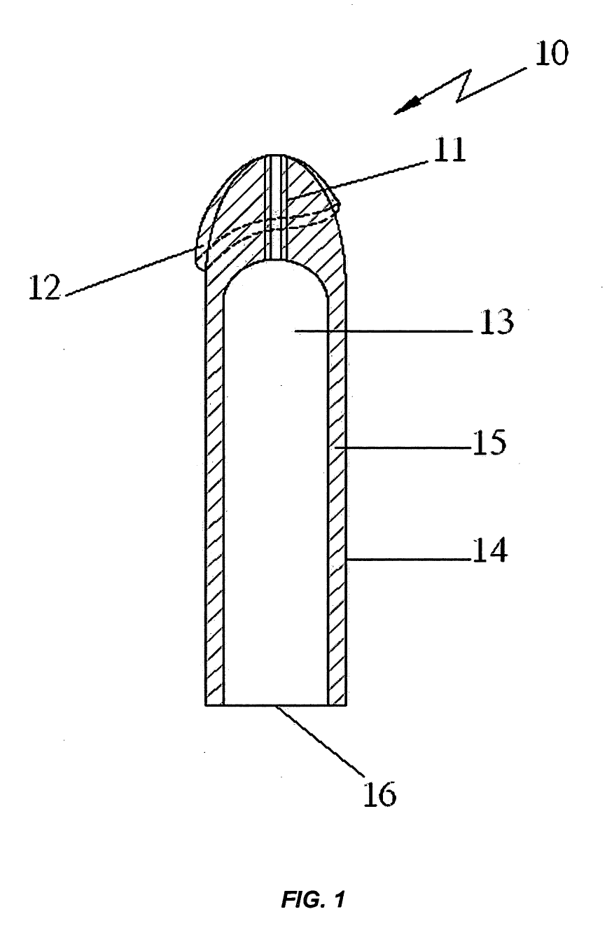

Referring to the drawings, the preferred embodiment of a prosthetic penile attachment/extension device that can be attached over an erect or semi-erect male phallus to enhance sexual intercourse, is illustrated and generally indicated as 10 in FIG. 1. The device 10 comprises an air passage 11, a head 12, an interior end 13, an exterior end 14, a sidewall 15, and an open end 16. The device 10 conforms more or less to the shape of a male phallus and presents a more natural look and feel.

The air passage 11 allows for the free passage of air between the interior 13 and exterior 14 of the device 10. All of the air within the sleeve of the device 10 escapes to the interior end 13, as the device 10 is pulled over an erect or semi-erect male phallus. This eliminates the existence of trapped air pockets that create soft spots. The head 12 of the device 10 is made up of a solid, rubber-like material that adds to the dimensions of length to the penis of the wearer.

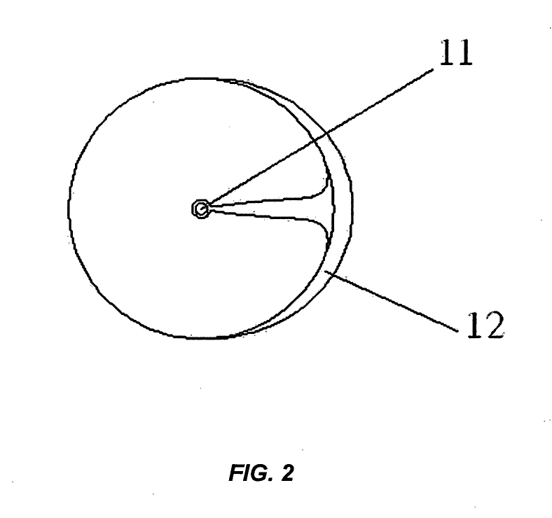

As shown in FIGS. 1 and 2, the interior 13 of the device 10 is open at the base end and closed at the anterior end by the head 12. Air is allowed to pass between the interior 13 and exterior 14 of the device by the addition of the air passage 11.

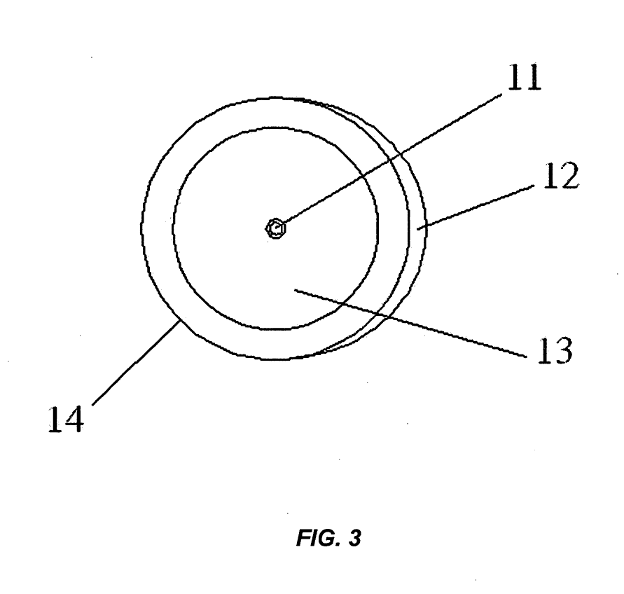

The sidewall 15, as seen in FIG. 3, is made of a stretchy, rubber-like material and is designed to fit tightly but comfortably around a male phallus. The sidewall 15 adds additional girth to the penis of the wearer. The open end 16 of the device 10 allows the wearer to insert the male phallus into the interior end 13 of the device.

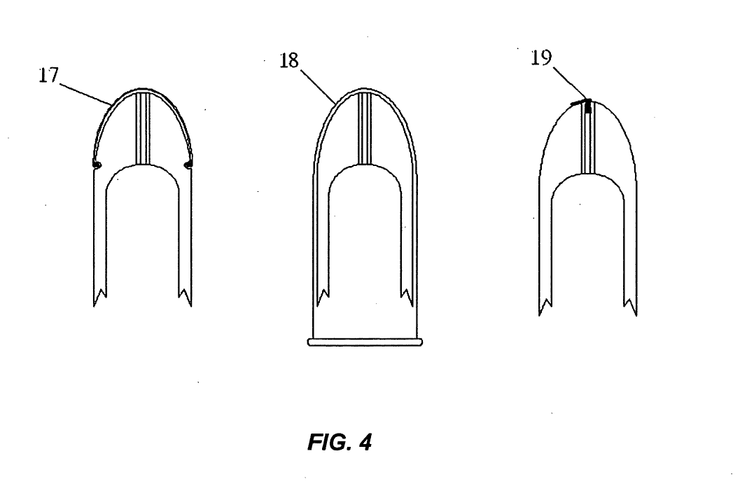

The air passage can be closed by the wearer by one of various methods described below and as illustrated in FIG. 4, thereby creating a vacuum within the device 10 that keeps it from falling off or slipping during use. The fitted, condom-like cap 17 is a method of sealing the air tube 11 of the device 10 after the wearer has inserted his erect or semi-erect phallus all the way to the interior end 13. It is made of a latex-like material that is thicker and more durable than that of a typical condom so it can be cleaned and used repeatedly. It is fitted with a tubular base that fits snuggly into a notch that follows the circumference of the head 12 of the device.

Another method is to use a common condom 18 to seal the air tube 11 by pulling it completely over the head 12 and sidewalls 15 of the device 10. Moreover, a specially fitted rubber or plastic stopper-like cap 19, as shown in FIG. 4, can be attached to the head 12 of the device 10 and inserted into the exterior end 14 of the air passage 11 to seal the air inside.

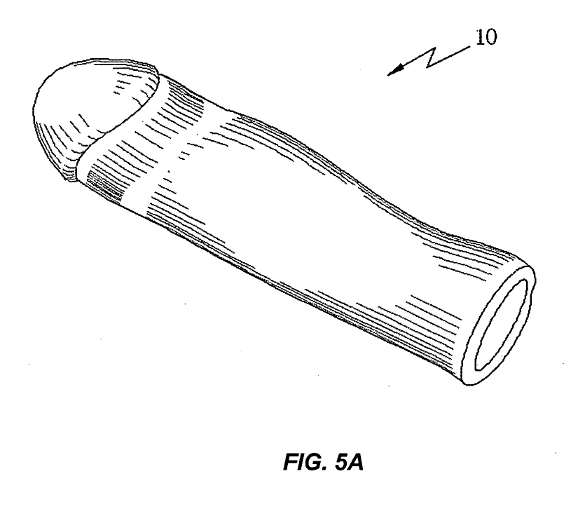

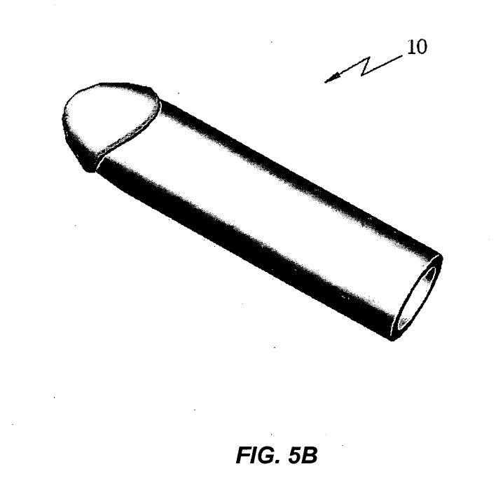

The operation and the alternate embodiment of the prosthetic penile attachment/extension device 10, as seen in FIG. 5, are briefly discussed herein. A male user with an erection inserts his penis into the open end 16 of the device 10 all the way to the head 12 of the device 10. The user then seals the air in the air passage 11 with either a fitted, condom-like cap 17, common condom 18, or stopper-like cap 19. The seal will create an ambient air pressure vacuum within the prosthetic device 10 that is sufficient to prevent it from slipping or falling off during use.

The material and shape of the prosthetic penile attachment/device 10 of the present invention is not limited to the aforementioned ones and can be made in various sizes and of different materials in the same manner and variety, that one sees in the many different types of penile extenders that are currently available in the market. Alternate methods of sealing the device 10 are also possible.

Alternatively, this invention could be provided in the form of a kit to allow for the modification of prior art penile extension devices. In this embodiment, a specially designed air tube would be provided to fit a particular prior art model/type along with a means to seal it.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. An external and improved penile prosthetic device that can be attached over an erect or semi-erect male phallus for use during sexual intercourse, comprising: a generally tubular exterior end having an open base end and a closed anterior end; the outer portion of the exterior end configured to resemble an erect male phallus; a generally tubular interior end having an open base end and a closed anterior end; an air passage fitted in between the exterior and interior ends along the longitudinal axis of the prosthetic device that allows for the free passage of air between the exterior and interior ends; a head portion on the exterior end that adds to the dimensions of length to the phallus of the wearer; and a sidewall of uniform or varying thickness that disposes the interior end spatially and coaxially inside the exterior end and sealed thereto at their open base ends to define a chamber therebetween, wherein said sidewall adds additional girth to the phallus of the wearer.

2. The external and improved penile prosthetic device of claim 1, wherein the head of the prosthetic device is made up of a solid, rubber-like material.

3. The external and improved penile prosthetic device of claim 1, wherein the sidewall is made of a stretchy, rubber-like material.

4. The external and improved penile prosthetic device of claim 3, wherein said sidewall is designed to fit tightly but comfortably around a male phallus.

5. A method of applying and securing an external and improved penile prosthetic device, said method comprising positioning over an erect or semi-erect male phallus a generally tubular shaped body having an exterior end and an interior end, which in turn have an open base end and a closed anterior end; a passage fitted in between the exterior and interior ends along the longitudinal axis of the prosthetic device that allows for the free passage of air between the exterior and interior ends; a head portion on the exterior end that adds to the dimensions of length to the phallus of the wearer; and a sidewall of uniform or varying thickness that disposes the interior end spatially and coaxially inside the exterior end and sealed thereto at their open base ends to define a chamber therebetween, wherein said sidewall adds additional girth to the phallus of the wearer.

6. The method of applying and securing an external and improved penile prosthetic device of claim 5, wherein the air passage is sealed with a fitted, condom-like cap having a tubular base that fits snuggly into a notch that follows the circumference of the head of the prosthetic device.

7. The method of applying and securing an external and improved penile prosthetic device of claim 6, wherein said condom-like cap is made of a latex-like material that is thicker and more durable than that of a typical condom, thereby facilitating the condom-like cap to be cleaned and used repeatedly.

8. The method of applying and securing an external and improved penile prosthetic device of claim 5, wherein the air passage is sealed with a traditional condom by simply pulling the condom over the head and sidewalls of the prosthetic device.

9. The method of applying and securing an external and improved penile prosthetic device of claim 5, wherein the air passage is sealed with a specially fitted stopper-like cap that can be attached to the head of the prosthetic device and inserted into the exterior end of the air tube to seal the air inside.

10. The method of applying and securing an external and improved penile prosthetic device of claim 9, wherein said stopper-like cap is made of rubber or plastic material.
